(19) United States
(12) Patent Application Publication
(10) Pub. No.: US 2005/0084062 A1
(43) Pub. Date: Apr. 21, 2005

(54) Magnification table and X-ray diagnostic device for mammography examinations

(76) Inventors: Jesper Andreasson, Jarfalla (SE); Stefan Karlsson, Sollentuna (SE)

Correspondence Address: SCHIFF HARDIN, LLP;PATENT DEPARTMENT, 6600 SEARS TOWER, CHICAGO, IL, 60606-6473, US

(21) Appl. No.: 10/922,008

(22) Filed: Aug. 19, 2004

(30) Foreign Application Priority Data
Aug. 19, 2003 (DE) 103 38 051.5

(51) Int. Cl. G21K 1/12; G01N 23/00; A61B 6/00; A61B 6/04

(52) U.S. Cl. 378/37

(57) ABSTRACT

A magnification table for mammography examinations has a table plate adapted to receive a subject thereon and a base. The table plate is substantially continuously vertically displaceable relative to the base, so that a magnification factor for radiograms of a female breast is continuously adjustable. An X-ray diagnostic device for mammography examinations has an arm supporting an X-ray tube, the arm having a longitudinal axis and a magnification table as described above, and an X-ray receiver. The table plate of the magnification table is displaceable in the direction of the longitudinal axis of the arm so that a magnification factor for radiograms of a female breast is continuously adjustable.

### BACKGROUND OF THE INVENTION

1. Field of the Invention

The present invention relates to a magnification table for mammography examinations with a plate on which an examination subject rests, and a base. The invention further relates to an X-ray diagnostic device for mammography examinations with an arm for an X-ray tube, a magnification table with a plate, an X-ray receiver (detector), and, between the X-ray tube and the magnification table, a compression plate that is connected to and displaceable along the arm.

2. Description of the Prior Art

Two magnification tables are shown for “MAMMOMAT 1000/3000/3000 Nova.” in a brochure by Siemens entitled “Accessories for Mammography.” A magnification factor of 1.5 can be achieved by means of one of the magnification tables, and a magnification factor of 1.8 can be achieved with an additional magnification table. X-ray receivers are integrated into the magnification tables at respective fixed distances from the table plate. One of the magnification tables with a desired magnification factor is fastened to the arm for the X-ray tube and thus replaces a subject table.

The brochure by Instrumentarium Imaging entitled “Alpha III: Take Your Mammography Into the 1990s” describes two magnification tables which, in connection with a subject magnification, can be fastened directly to the existing subject table of a mammography device in which an X-ray receiver is disposed. In these two magnification tables in the brochure as well, when the magnification table has been attached to the subject table, the distance between the table plate and the X-ray receiver is fixed, and consequently only two defined subject magnifications can be performed.

A disadvantage of these known magnification tables is that the selection of the subject magnifications is very limited. Another disadvantage is that there must be a separate magnification table for each degree of subject magnification. This is relatively expensive. Moreover, it is necessary for a user to exchange magnification tables whenever the magnification of the subject is changed.

### SUMMARY OF THE INVENTION

An object of the present invention is to provide a magnification table of the above-described type, and an X-ray diagnostic device having such a magnification table, that are relatively simple in construction and therefore inexpensive and with which it is possible to set nearly any arbitrary magnification factor.

The object is inventively achieved by a magnification table for mammography examinations having a table plate and a base, the table plate being substantially continuously vertically displaceable relative to said base. The vertical adjustability of the table plate relative to the base of the magnification table makes possible a nearly arbitrary continuous adjustment (i.e. not limited to steps or a number of fixed positions) of the distance of the table plate from the base of the magnification table, so that a nearly arbitrary setting of the magnification factor is possible.

According to an embodiment of the invention, the magnification table has a two-part telescoping structure the first part of which includes the base and the second part of which carries the table plate, with the second part being displaceable vertically relative to the first part. The result is a relatively simple and inexpensive construction of the magnification table.

In a variant of the invention, the magnification table contains an X-ray detector.

The above object is also inventively achieved by an X-ray diagnostic device with a magnification table having a table plate adjustable in the direction of the longitudinal axis of the support arm for the x-ray tube such that the distance between the table plate of the magnification table and the X-ray receiver (detector) can be modified. Based on this construction of the magnification table, a nearly arbitrary continuous adjustment of the distance of the table plate of the magnification table from the X-ray receiver can be performed, so that a nearly arbitrary setting of the magnification factor is possible.

In an embodiment, the X-ray diagnostic device has a subject table that is disposed at the arm that supports the X-ray tube and that contains the X-ray receiver, and the magnification table has a two-part telescoping structure, with a first part disposed at the subject table, and a second part carrying the table plate and being displaceable relative to the first part.

In another embodiment of the X-ray diagnostic device, the magnification table likewise has a two-part telescopic structure, but the first part is disposed at the arm for the X-ray tube and contains the X-ray receiver. The second part carries the table plate and is again displaceable relative to the first part.

### DESCRIPTION OF THE DRAWINGS

FIG. 1 is a side view of an X-ray diagnostic device for mammography examinations having a magnification table according to the invention.

FIG. 2 is a side view of the magnification table according to FIG. 1.

FIG. 3 shows the magnification table according to FIGS. 1 and 2, seen from the patient's perspective.

### DESCRIPTION OF THE PREFERRED EMBODIMENTS

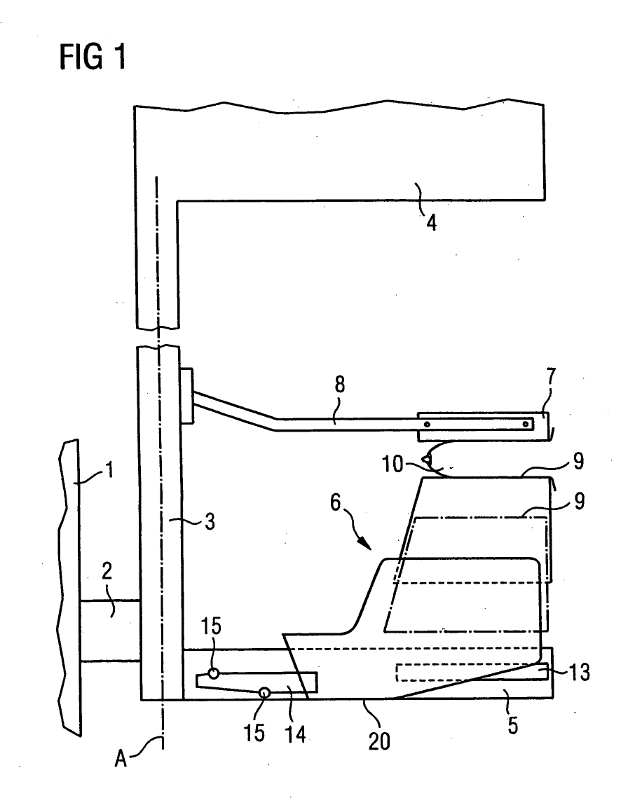

FIG. 1 is a side view of an X-ray diagnostic device for mammography examinations with part of a device stand 1 that is connected in rotating fashion by a shaft 2 to an arm 3, which has a longitudinal axis A, for an X-ray tube 4, and to a subject table 5. In this embodiment, an X-ray receiver 13 in the form of a film cartridge is disposed in the subject table 5. In another embodiment, the X-ray receiver 13 can be a digital image plate detector. Attached on the subject table 5 is a magnification table 6, which is described below. Disposed between the X-ray tube 4 and the table plate 9 of the magnification table 6 is a compression plate 7, which is connected to the arm 3 by way of a bracket 8 and is displaceable along said arm.

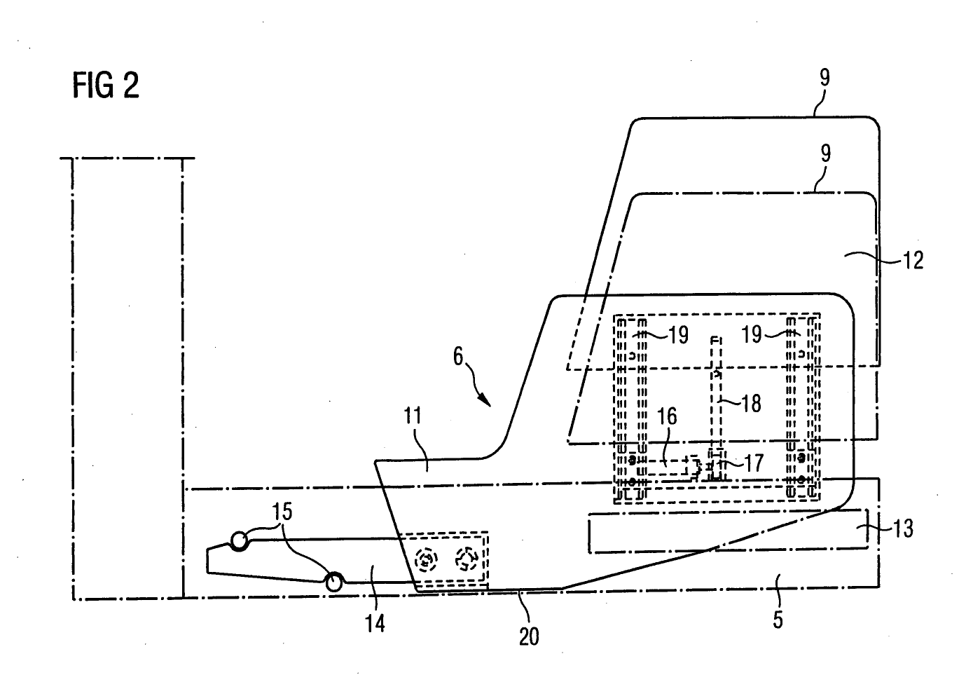

FIG. 2 illustrates the magnification table 6. The magnification table 6 has a two-part telescopic configuration, wherein the first part 11, which has a base a base 20, is attachable to the subject table 5 and is removable therefrom. The fastening device for the first part 11 of the magnification table 6 has arms 14 that are fixed to the first part 11 and that extend along the two sides of the subject table 5, though only one arm 14 is shown in FIG. 1. The arms 14 are held by pegs 15 that are fixed to the subject table 5. The second part 12 of the magnification table 6 carries the abovementioned table plate 9. The second part 12, and thus the table plate 9 of the magnification table 6 as well, are vertically displaceable substantially in the direction of the longitudinal axis A of the arm 3. The displacement of the second part 12 occurs with the aid of at least one motor 16 that drives a gearwheel 17. The motor 16 with the gearwheel 17 is disposed in the first part 11 of the magnification table 6. The second part 12 of the magnification table is provided with a gear rod 18 that is disposed in front of the gearwheel, whereby the gear rod 18 and the gearwheel 17 mesh. In order to stabilize the displacement of the second part 12 relative to the first part 11, the second part 12 and the first part 11 are provided with interlocking rails 19. In order to displace the second part 12, a user activates the motor 16 with the aid of known operating elements, which drive the gearwheel 17 so that the gear rod 18 and therefore the second part 12 with the table plate 9 are moved in the direction of the longitudinal axis A of the arm 3. That way, the distance between the table plate 9 and the X-ray receiver 13 can be freely selected, so that a desired magnification factor can be achieved in a radiogram of a breast. The highest position of the table plate 9 is represented in FIG. 2 by a solid line, and the lowest position of the table plate 9 is represented by a dotted line.

FIG. 1 shows a breast 10 that is to be examined. The breast 10 rests on the table plate 9 and is compressed by the compression plate 7 prior to imaging. In FIG. 1 as well, the highest and lowest positions of the table plate 9 are represented by solid and dotted lines, respectively.

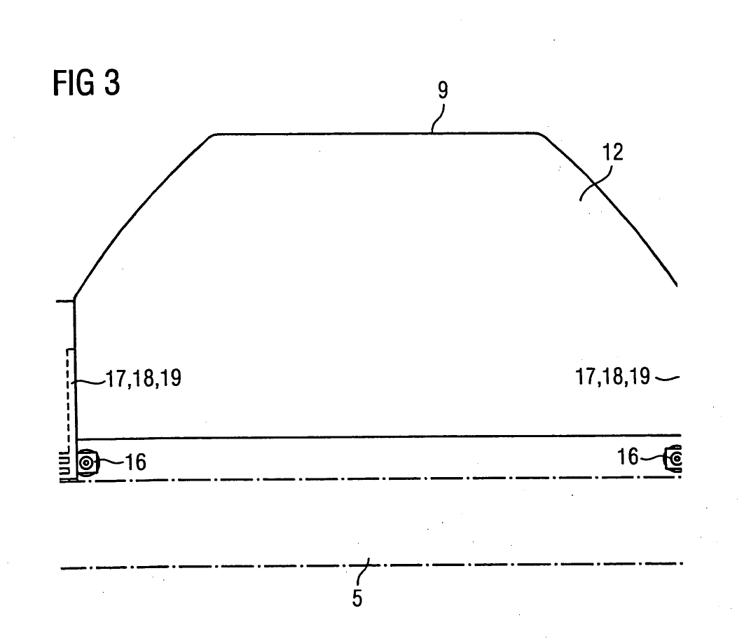

FIG. 3 shows the magnification table 6 from the patient's perspective. FIG. 3 shows that the first part 11 of the magnification table 6 is somewhat wider than the width of the subject table 5, so that the first part 11 is disposed in stable fashion at the subject table 5 and its sides. FIG. 3 also shows that the second part 12 is partly disposed in the first part 11 and can be displaced relative to this part 11. As also can be seen from FIG. 3, the magnification table 6 is expediently provided on both sides with a motor 16, a gearwheel 17, a gear rod 18 and rails 19 as described above, which cannot be seen in FIG. 1. The result is a stable and reliable displacement of the second part 12 and the table plate 9 thereof.

The distance between the table plate 9 and the X-ray receiver 13 can be automatically calculated by a device control, for instance a computer (not shown), with the aid of position sensors among other things, and the magnification factor, which depends on this distance, can be read from a display that is connected to the computer.

A nearly arbitrary magnification factor can be set by means of the inventive magnification table 6 with its continuous adjustment of the distance between the table plate 9 and the X-ray receiver 13.

Within the scope of the invention, the inventive magnification table can replace the subject table 5 of the X-ray diagnostic device. In this case, the magnification table is disposed at the arm 3 of the X-ray diagnostic device, and an X-ray receiver is integrated into the first part of the magnification table. Mounting of this magnification table at the arm 3 can be accomplished with arms 14 and pegs 15 in the same manner as was previously described for the attaching of the magnification table to the subject table 5.

Although modifications and changes may be suggested by those skilled in the art, it is the intention of the inventors to embody within the patent warranted hereon all changes and modifications as reasonably and properly come within the scope of their contribution to the art.

1. A magnification table for mammography examinations, comprising:
a table plate adapted to receive an examination subject thereon;
a base; and
an adjustable connection between said table plate and said base allowing substantially continuous vertical displacement of said table plate relative to said base.

2. A magnification table as claimed in claim 1 comprising a first part including said base and a second part carrying said table plate, and wherein said adjustable connection comprises a telescoping connection between said first part and said second part.

3. A magnification table as claimed in claim 1 comprising an X-ray receiver disposed at a location beneath said table plate and above a bottom of said base.

4. An X-ray diagnostic device for mammography examinations comprising:
an X-ray tube;
an arm supporting said X-ray tube, said arm having a longitudinal axis;
a magnification table having a table plate adapted to receive an examination subject thereon;
an X-ray receiver; and
an adjustment arrangement connected to said table plate allowing substantially continuous adjustment of said table plate along said longitudinal axis of said arm for substantially arbitrarily varying a distance between said table plate and said X-ray receiver.

5. An X-ray diagnostic device as claimed in claim 4 comprising a subject table carried by said arm and containing said X-ray receiver, and wherein said magnification table comprises a first part disposed at said subject table and a second part carrying said table plate, and a telescoping connection between said first part and said second part allowing substantially continuous displacement of said second part relative to said first part in said direction of said longitudinal axis.

6. An X-ray diagnostic device as claimed in claim 4 wherein said magnification table comprises a first part containing said X-ray receiver and mounted at said arm, and a second part carrying said table plate, and a telescoping connection between said first part and said second part allowing substantially continuous displacement of said second part relative to said first part in said direction of said longitudinal axis of said arm.
